(19) United States
(12) Patent Application Publication
(10) Pub. No.: US 2025/0381373 A1
(43) Pub. Date: Dec. 18, 2025

(54) MEDICAL SYSTEMS, DEVICES, AND RELATED METHODS FOR DELIVERY OF FLUIDS

(71) Applicant: BOSTON SCIENTIFIC MEDICAL DEVICE LIMITED, Galway (IE)

(72) Inventor: Deepak Kumar SHARMA, Muzaffarnagar (IN)

(73) Assignee: BOSTON SCIENTIFIC MEDICAL DEVICE LIMITED, Galway (IE)

(21) Appl. No.: 19/240,273

(22) Filed: Jun. 17, 2025

Related U.S. Application Data
(60) Provisional application No. 63/661,084, filed on Jun. 18, 2024.

(51) Int. Cl. A61M 25/10; A61M 25/00

(52) U.S. Cl. CPC A61M 25/10181; A61M 25/0023

(57) ABSTRACT

A medical device may include an outer tube and a deformable member disposed within the outer tube. A gap may be disposed between the outer tube and the deformable member. The medical device may further include a sheath. The sheath may include an opening on a distal portion of the sheath. The medical device may include a port configured to supply a fluid to the gap. The fluid may be configured to deform the deformable member radially inwardly to compress the sheath and deliver a second fluid within the sheath out of the opening.

### CROSS-REFERENCE TO RELATED APPLICATION(S)

This patent application claims the benefit of priority to U.S. Provisional Application No. 63/661,084, filed on Jun. 18, 2024, which is incorporated by reference herein in its entirety.

### TECHNICAL FIELD

The disclosure relates generally to systems, devices, and methods for delivering one or more fluids. More specifically, aspects of the disclosure pertain to systems, devices, and/or methods for delivering viscous fluid that may include one or more treatment agents to a target site.

### BACKGROUND

Bleeding ulcers or other wound sites may occur, for example, in a subject's gastrointestinal (GI) tract. For example, following another diagnostic or treatment procedure, such as endoscopic mucosal resection (EMR) or endoscopic submucosal dissection (ESD), bleeding may be desired to be prevented or treated. In another example, a line of sutures in a GI tract may be in need of reinforcement. In a further example, a fistula may require treatment. In yet another example, peroral endoscopic myotomy (POEM) sites may need closure. Therefore, a need exists for systems, devices, and methods for delivering one or more fluids.

### SUMMARY

The disclosure includes systems, devices, and methods for delivering one or more fluids, such as treatment agents, to a target site of a subject to, for example, help heal an ulcer and/or to perform hemostasis. Each of the aspects disclosed herein may include one or more of the features described in connection with any of the other disclosed aspects.

A medical device may include an outer tube and a deformable member disposed within the outer tube. A gap may be disposed between the outer tube and the deformable member. The medical device may further include a sheath. The sheath may include an opening on a distal portion of the sheath. The medical device may include a port configured to supply a fluid to the gap. The fluid may be configured to deform the deformable member radially inwardly to compress the sheath and deliver a second fluid within the sheath out of the opening.

Any of the systems, devices, and methods disclosed herein may include any of the following features. The sheath may be disposed within a lumen defined by the deformable member. The medical device may include a hub affixed to a proximal portion of the outer tube. The medical device may further include a tubular member affixed to the deformable member and the hub. The medical device may further include a fluid port extending from the hub. The fluid port may include a lumen. The lumen may be in fluid communication with the gap. The fluid port may form an obtuse angle relative to the outer tube. The medical device may further include a cap affixed to a distalmost portion of the outer tube and a distalmost portion of the deformable member. The opening may be disposed proximally of a distalmost end the cap. The gap may terminate distally at a proximalmost surface of the cap. The sheath may include a tapered portion proximal of the opening. The fluid may be a first fluid, and the sheath may include a second fluid within a lumen of the sheath. Compression of the sheath may cause the second fluid to be ejected from the opening. Compression of the sheath may cause the medical device to transition from a first configuration to a second configuration. The second configuration may include a smaller volume of the second fluid than the first configuration. In the second configuration, at least a portion of the gap may have a larger radial width than in the first configuration. The deformable member may be a balloon.

A medical device may include an outer tube, a deformable member disposed radially within the outer tube, a gap disposed radially between the outer tube and the deformable member, and a sheath. The sheath may include a first fluid. The sheath may be radially within the deformable member. The gap may be configured to receive a second fluid from a fluid source. The second fluid may be configured to deform the deformable member so that the deformable member compresses the sheath to deliver the first fluid through an opening of the sheath.

Any of the systems, devices, and methods disclosed herein may include any of the following features. The medical device may further include a hub. The hub may include a fluid port that may be configured to couple to the fluid source. The fluid port may be in fluid communication with the gap. The medical device may further include a cap coupled to a distal end of the outer tube and a distal end of the deformable member.

A medical device may include a proximal hub. The proximal hub may include a fluid port and a shaft extending distally from the hub. The shaft may include a balloon and a sheath containing a first fluid. The sheath may be disposed within a lumen of the balloon. Delivery of a second fluid into the fluid port may be configured to contact a radially outer surface of the balloon to cause the balloon to deform radially inward to compress the sheath and deliver the first fluid from an opening of the sheath.

Any of the systems, devices, and methods disclosed herein may include any of the following features. The shaft may include an outer tube. The balloon may be disposed within the outer tube. A gap may be formed between the radially outer surface of the balloon and a radially inner surface of the outer tube. The fluid port may be in fluid communication with the gap.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of a proximal portion of an exemplary medical device in a first configuration.

FIG. 2 depicts a perspective view of a distal portion of the exemplary medical device of FIG. 1.

FIG. 3 depicts a perspective view of the proximal portion of the exemplary medical device of FIG. 1 in a second configuration.

FIG. 4 depicts a perspective view of the proximal portion of the exemplary medical device of FIG. 1 in a third configuration.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

A fluid may be used to prevent or treat bleeding or other conditions in a GI tract. In examples, a viscous fluid (e.g., a highly viscous fluid) may be delivered to a wound or other treatment site to form a protective layer that helps to treat or minimize delayed bleeds, potential perforations, and stricture formations. Conventional devices for fluid delivery may include one or more fluid channels, with the one or more fluid channels each having a small diameter. Viscous fluids may require a large amount of force or pressure to flow through narrow fluid channels. Applying a large amount of force or pressure to urge the viscous fluid distally may be difficult and/or time-consuming for the user. Additionally, applying the large amount of force or pressure may increase the risk of one or more components of the medical device deforming, breaking, or otherwise failing.

Aspects of this disclosure seek to improve and ease a user's ability to deliver a highly viscous fluid via a medical device. In some examples, the medical device may be delivered to a target site through a working channel of an insertion device, such as a medical scope (e.g., an endoscope). Additionally, various aspects of this disclosure may help the user perform wound treatment, prophylactic treatment, and/or hemostasis within the subject, reduce overall procedure time, reduce overall procedure costs, etc. Embodiments of this disclosure are configured to deliver viscous fluid when a pressurized fluid (e.g., air or a liquid) is applied to an inflatable feature (e.g., a balloon or bladder). The presence of an inflatable feature for fluid delivery, as opposed to a push feature for fluid delivery, may allow for an easier application of force and/or a sufficient amount of force to deliver viscous fluid through the various openings and channels of the embodiments of this disclosure. Furthermore, such fluids may be disposed at a distal end of the device and may not be required to travel through a full length of the medical device (e.g., through a length of the medical device inserted through a working channel of an insertion device). While the disclosure primarily relates to highly viscous fluids on the order of 200 centipoise or greater, one of ordinary skill in the art that the disclosure may be applicable to fluids of any viscosity.

Examples of fluids (e.g., biocompatible viscous fluids) that may be delivered using the devices disclosed herein include, but are not limited to, fibrin, thrombin, fluids including calcium salts, cyanoacrylates, albumin and glutaraldehyde, poly (ethylene glycol) (PEG), polyurethane, etc. Such fluids may be endoscopically delivered adhesives or other agents that help to create a protective layer that minimizes delayed bleeds, potential perforations, and stricture formations. However, the list above is merely exemplary, and the disclosed device may be used to deliver a range of fluids.

One of ordinary skill in the art will appreciate that the systems, devices, and methods of this disclosure may be used with a variety of biocompatible fluids and that the systems, devices, and methods of the disclosure may be applicable to various medical procedures beyond bleeding control.

Various aspects of this disclosure also relate to delivery and/or mixing of various hemostatic agents at a treatment site. For example, a user may deploy multiple different hemostatic agents at the target treatment site via the systems, devices, and methods disclosed herein. In an example, a first agent (Part A) and a second agent (Part B) may be mixed together in a predefined ratio at the target treatment site. Part A and Part B may crosslink when they come in contact with one another. The cross-linked structure of the resulting combination agent (A+B) may have enhanced hemostatic properties compared to agents A or B individually. In a non-limiting example, Part A may be fibrinogen (e.g., lyophilised pooled human concentrate) and Part B may be thrombin (e.g., of bovine or human origin). Fibrinogen and thrombin may be mixed immediately before application at a target treatment site. The biocompatible fibrinogen and thrombin mixture may also contain calcium salts. One of ordinary skill in the art will appreciate that such a hemostatic mixture may be used at various locations in the human body, such as the stomach, esophagus, and colon.

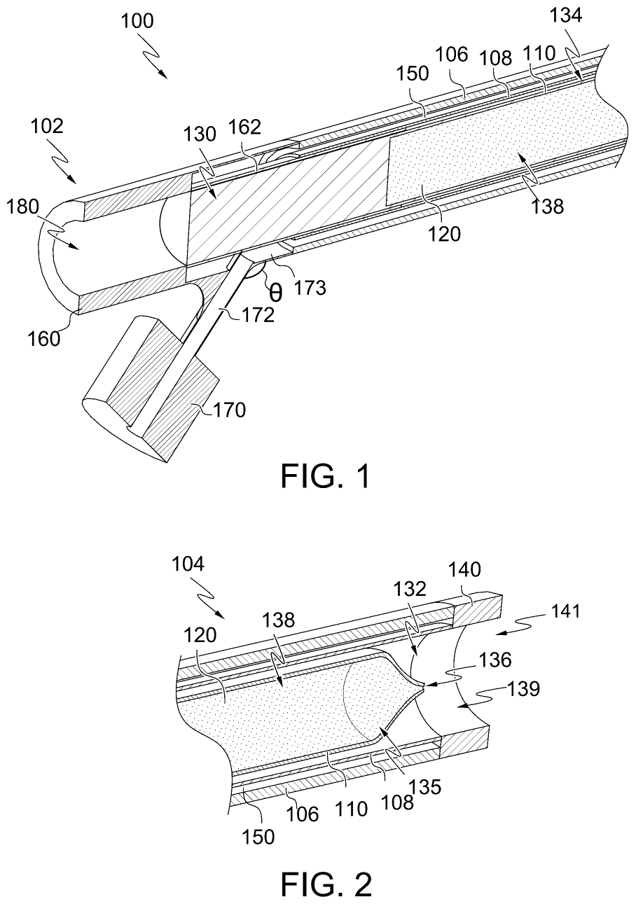

FIG. 1 depicts a perspective view of a proximal portion 102 of an exemplary medical device 100 in a first, undeployed, configuration, and FIG. 2 depicts a distal portion 104 of the medical device 100 in the first, undeployed configuration. Medical device 100 may be delivered to a target site via an endoscope or other similar scope devices. Medical device 100 may be inserted into a working channel of an endoscope to be deployed at a target site inside of a patient, such as at an ulcer. Alternatively, medical device 100 may be delivered directly to a target site without use of a delivery device. A user may operate medical device 100 to control the internal bleeding of a patient via application of viscous hemostatic agents to the bleed. Medical device 100 may require substantially less force to be exerted by the user to deploy highly viscous fluids as compared to conventional devices. Medical device 100 may supply fluid to an inflatable portion (e.g., a bladder or balloon) in order to squeeze (e.g., apply pressure to) a sheath (e.g., a tube) containing an agent to deploy the agent. While reference is made to highly viscous fluids, one of ordinary skill in the art will recognize that the systems, devices, and methods of this disclosure are applicable to fluids with various viscosities.

As shown in FIGS. 1 and 2, medical device 100 may include a proximal portion 102 and a distal portion 104. Distal portion 104 may be an insertion portion (e.g., a shaft) that is inserted into a body lumen (e.g., via an insertion device). Medical device 100 may include an outer tube 106. Outer tube 106 may be flexible and may have a length configured to extend through a delivery device. Outer tube 106 may radially surround (e.g., extend radially outwards of) a balloon 108, and a sheath 110. In other words, balloon 108 and sheath 110 may be disposed within a central lumen of outer tube 106. Sheath 110 may contain an agent 120, which may be a fluid. Alternatively, sheath 110 may contain another type of fluid.

Medical device 100 may include a sheath 110. Sheath 110 may be flexible and deform in response to pressure applied via balloon 108, as discussed below. Sheath 110 may have a proximal portion 130 (FIG. 1) a distal portion (FIG. 2) and a middle portion 134 between proximal portion 130 and distal portion 132. Sheath 110 may include/define a lumen 138, which may extend through at least middle portion 134 and distal portion 134. Lumen 138 may be configured to contain an agent 120. In some examples, medical device 100 may be pre-loaded with agent 120; in alternatives, agent 120 may be loaded by a user prior to use of medical device 100. Agent 120 may comprise one or more biocompatible fluids discussed above. In the first, undeployed configuration of FIGS. 1 and 2, sheath 110 may have an approximately uniform diameter/width along proximal portion 130 and middle portion 134.

In some aspects, lumen 138 may include a partition separating two or more fluids. The partition may extend from a proximal inner face of sheath 110 along the longitudinal axis of lumen 138 and may terminate at opening 136. Each partitioned portion of lumen 138 may contain a fluid (e.g., subcomponents of a combination agent). Expelling multiple fluids from opening 136 may allow the multiple fluids to mix and cure at the target site. In some aspects, the subcomponents of a combination agent may be mixed before filing lumen 138 with the resulting combination agent via a port (not shown in FIG. 2).

Proximal portion 130 of sheath 110 may have a closed proximalmost end. In some examples, proximal portion 130 may have a solid cross-section (not define lumen 138). Such a configuration may provide support and/or additional structure to sheath 110 and/or to other elements of proximal portion 102, discussed below.

Distal portion 132 of sheath 110 (and lumen 138) may terminate distally at distal opening 136 (e.g., sheath 110 may include distal opening 136 on distal portion 132). Distal portion 132 may include a tapered portion 135 proximal of opening 136. Tapered portion 135 may taper radially inward in a distal direction. A diameter/width of opening 136 may be less than a diameter of sheath 110 proximal of tapered portion 135 (e.g., at middle portion 134). A size of opening 136 may be configured to deliver a desired amount of agent 120 at a desired velocity. Opening 136 may allow agent 120 to be ejected out of sheath 110 when pressure is applied to sheath 110 and lumen 138 by balloon 108, as discussed below.

Balloon 108 may at least partially surround sheath 110 and may be disposed within outer tube 106 (e.g., within a central lumen defined by outer tube 106). Balloon 108 may be formed from a flexible (e.g., elastic) material. Balloon 108 may be approximately tubular shaped and may define a central lumen 139, as most clearly shown in FIG. 2. However, balloon 108 may have alternative shapes. For example, balloon 108 may surround only a portion of sheath 110 (e.g., may be to one side of sheath 110). In such examples, balloon 108 may be approximately tubular or may be a sheet of flexible material that is coupled to inner surfaces of outer tube 106.

As shown in FIG. 2, a cap 140 may couple a distalmost end of balloon 108 to a distalmost end of outer tube 106. Cap 140 may have a substantially annular shape, such that lumen 139 has a distalmost opening 141. Sheath 110 may be disposed in lumen 139, such that opening 136 of sheath 110 is in fluid communication with distalmost opening 141 of lumen 139. Opening 136 may be positioned proximally of a distalmost end of cap 140. Cap 140 may be positioned distally of the distalmost portions of tube 106 and balloon 108. In some aspects, distal portion 132 and opening 136 may extend distally of cap 140 (e.g., such that opening 136 may be exposed at the target site distally of cap 140).

A gap 150 (e.g., a channel or a lumen) may be defined radially between balloon 108 and outer tube 106 (e.g., between a radially outer surface of balloon 108 and a radially inner surface of outer tube 106). A proximal surface of cap 140 may define a closed distalmost end of gap 150. As described in further detail below, a fluid may be delivered into gap 150 in order to compress balloon 108. In the first, undeployed configurations of FIGS. 1 and 2, gap 150 may have an approximately uniform thickness proximally of tapered portion 135.

Proximal portion 102 may include a hub 160. Hub 160 may be affixed to a proximal end of outer tube 106. Medical device 100 may further include a tube 162. Tube 162 may couple balloon 108 to hub 160. Tube 162 may be any tubular member. For example, a proximal portion of balloon 108 may extend around a distal portion tube 162, or a distal portion of tube 162 may extend around balloon 108. Tube 162 and balloon 108 may be secured (affixed) to one another with, for example, adhesive. A proximal portion of tube 162 may be coupled to hub 160 (e.g., by adhesive, molding, or any other mechanism). In some examples, tube 162 may be integrally formed with hub 160 from a single piece of material. A radially outer wall of hub 160 (coupled to outer tube 106) may extend distally of more radially inner portions of hub 160 that are coupled to tube 162. Tube 162 may terminate distally at a position distal of a proximalmost portion of sheath 110. In examples, at least a portion of proximal portion 130 of sheath 110 may extend through tube 162.

A fluid port 170 may be disposed (e.g., coupled to or formed integrally with) on hub 160. Lumen 172 may extend through fluid port 170 and may have a proximalmost open end. Fluid port 170 may be configured to couple to a fluid source (e.g., a syringe device, source of compressed air, or water bottle). Fluid port 170 may be configured with various neck widths and thread finishes such that fluid port 170 is compatible with (e.g., can mate with) a variety of fluid sources. Lumen 172 may be in fluid communication with gap 150. For example, lumen 172 may be in fluid communication with a channel 173 between an outer wall of port 170 (e.g., an outer wall coupled to outer tube 105) and tube 162. Channel 173 may be in fluid communication with gap 150. However, in some examples, tube 162 may be omitted, and gap 150 may extend proximally to port 170. Hub 160 may form a closed proximalmost end of gap 150 and/or channel 173.

Fluids supplied to lumen 172 via fluid port 170 may include, for example, air, water, or saline. One of ordinary skill in the art will appreciate that various other fluids may be supplied to port 170 without departing from the scope of the disclosure. In some aspects, different fluids may be supplied during a medical procedure. Fluid supplied via a fluid port 170 through lumen 172 may flow into gap 150 and press walls of balloon 108 radially inward, as discussed below. Fluid port 170 may be affixed to a remainder of hub 160 160 at an angle θ, which may be approximately 135 degrees with respect to tube 106, although such an angle is merely exemplary. An angle may be selected for ease of fluid flow.

Hub 160 may include a proximalmost end 180. Proximalmost end 180 is depicted in FIG. 1 with a proximal open end, although proximalmost end 180 may also be closed. In configurations where proximalmost end 180 is at least partially open, proximalmost end 180 may be configured to deliver agent 120 to sheath 110 or to receive a sheath 110 that is packaged separately from a remainder of medical device 100. In some aspects, proximalmost end 180 may be closed (e.g., sealed) such that proximalmost end 180 terminates proximally with a proximalmost face (not shown in FIG. 1). In such examples, medical device 100 may be pre-loaded with agent 120 and/or sheath 110. Alternatively, agent 120 and/or sheath 110 may be back-fed through distalmost opening 141 of lumen 139.

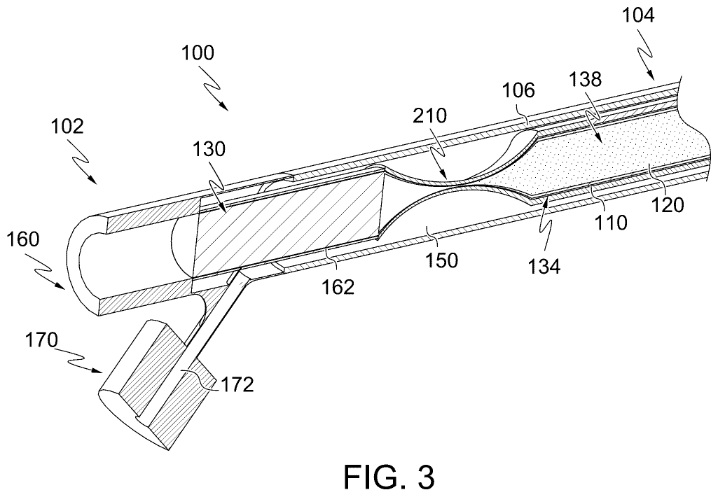

FIG. 3 depicts a proximal portion of medical device 100 in a second, partially deployed state. FIG. 3 depicts medical device 100 during an intermediate state of an inflation of balloon 108, with a portion of sheath 110 being compressed by balloon 108. To transition from the first configuration of FIGS. 1 and 2 to the second configuration of FIG. 3, fluid may be delivered into gap 150 via lumen 172.

A user may begin to deliver agent 120 from medical device 100 by delivering a fluid into lumen 172 via fluid port 170, using any suitable fluid source. Fluid may flow through lumen 172 into gap 150. After moving through lumen 172, fluid may enter channel 173 and/or gap 150 at angle θ, which may be any suitable angle. Fluid may be directed distally due to angle θ being obtuse relative to outer tube 106. As discussed above, gap 150 and/or channel 173 may terminate proximally at hub 160, thereby preventing proximal flow of fluid.

Fluid in gap 150 (between balloon 108 and outer tube 106) may exert a radially inward force on balloon 108. Fluid in gap 150 may be radially outward of balloon 108 and may contact a radially outer surface of balloon 108. Balloon 108 may be composed of a flexible material, such as an elastomer, and outer tube 106 may be relatively less flexible (e.g., having a larger elastic modulus) as compared to balloon 108. Balloon 108 may thus elastically deform or otherwise move (e.g., move radially inward, toward a central longitudinal axis of medical device 100) in response to fluid (e.g., air) pressure in gap 150, while outer tube may not bulge outward. As discussed above, fluid may be unable to travel proximally of hub 160. Similarly, fluid may be unable to travel distally of cap 140. With these proximal and distal portions of gap 150 sealed, fluid pressure may begin to build in gap 150 as fluid is supplied through fluid port 170 and lumen 172. Increasing fluid pressure may cause balloon 108 to elastically deform (e.g., radially inward). In turn, a size of gap 150 may increase. Balloon 108 may also be referred to an inflatable (or expandable) portion/member or a deformable portion/member.

Expansion of balloon 108 may apply a squeezing force to sheath 110 (and lumen 138). The compressive (e.g., squeezing) force may generally be directed radially inward towards a longitudinal axis defined by sheath 110. The compressive force applied to sheath 110 may cause compressive deformation of sheath 110, similar to squeezing a tube of toothpaste. As the compressive force increases, a compressed portion 210 of sheath 110 may form. Because lumen 172 is in fluid communication with a proximal portion of gap 150, compressed portion 210 may initially form at a proximal end of middle portion 134 of sheath because proximal portions of balloon 108 may be the first portions to deform radially inwardly. Proximal portion 130 of sheath 110 may be proximal of balloon 108 and may have a solid cross-section and thus may not deform into compressed portion 210. Compressed portion 210 of sheath 110 and corresponding portions of balloon 108 may have an arcuate cross-sectional shape. A size of gap 150 may increase in radial thickness at compressed portion 210.

Growth of compressed portion 210 (distal progression of compressed portion 210) may correspond with agent 120 being ejected or otherwise dispersed out of opening 136. For each change in volume of sheath 110, an approximately corresponding volume of agent 120 may be ejected from opening 136 at the target site. Thus, lumen 138 may include a smaller volume of agent 120 in the second configuration than in the first configuration. A user may increase or decrease the rate of dispersal of agent 120 by modifying the fluid (e.g., fluid) flow rate into medical device 100. For example, a user may wish to slow the release of agent 120 by reducing the fluid flow into medical device 100. Similarly, a user may wish to temporarily halt the dispersal of agent 120 by ceasing fluid flow into medical device 100 (e.g., closing a valve at a fluid source). A user may view the dispersal of agent 120 via an imaging device affixed to the insertion device (e.g., a camera affixed to a distal tip of an endoscope). A user may also track how much agent 120 has been delivered by monitoring a fluid pressure via fluid port 170 and/or a volume of fluid delivered.

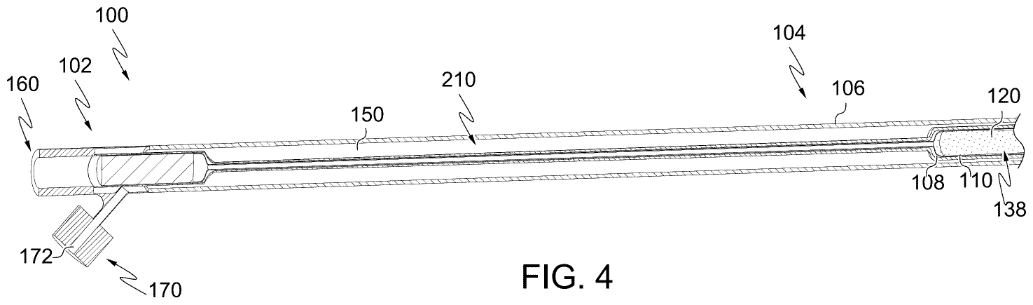

FIG. 4 depicts proximal portion 102 of medical device 100 in a third, further deployed state. FIG. 4 depicts compressed portion 210 of sheath 110 in a state of greater compression than depicted in FIG. 3, corresponding to a greater volume of dispersed agent 120 and a greater amount of fluid delivered through lumen 172. To transition from the second configuration to the third configuration, further fluid may be delivered into gap 150. As compared with the second configuration of FIG. 3, compressed portion 210 extends further distally along middle portion 134 of sheath 110 due to inward deformation of increasingly distal portions of balloon 108. In other words, compressed portion 210 occupies a greater proportion of sheath 110. Gap 150 may increase in radial thickness along compressed portion 210. A user may continue to apply fluid to fluid port 170 and lumen 172 until a medical procedure is completed (e.g., a medical procedure requiring less than the entire volume of agent 120) or all the dispersible volume of agent 120 is ejected through opening 136. As discussed above, a fluid pressure at fluid port 170 and/or a volume of fluid delivered into port 170 may be indicative to a user of how much agent 120 has been delivered. Lumen 138 may contain a smaller volume of agent 120 in the third configuration than in the second configuration.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
an outer tube;
a deformable member disposed within the outer tube, wherein a gap is disposed between the outer tube and the deformable member;
a sheath including an opening on a distal portion of the sheath; and
a port configured to supply a fluid to the gap, wherein the fluid is configured to deform the deformable member radially inwardly to compress the sheath and deliver a second fluid within the sheath out of the opening.

2. The medical device of claim 1, wherein the sheath is disposed within a lumen defined by the deformable member.

3. The medical device of claim 2, further comprising:
a hub affixed to a proximal portion of the outer tube.

4. The medical device of claim 3, further comprising:
a tubular member affixed to the deformable member and the hub.

5. The medical device of claim 3, further comprising:
a fluid port extending from the hub, wherein the fluid port includes a lumen, wherein the lumen is in fluid communication with the gap.

6. The medical device of claim 5, wherein the fluid port forms an obtuse angle relative to the outer tube.

7. The medical device of claim 1, further comprising:
a cap affixed to a distalmost portion of the outer tube and a distalmost portion of the deformable member.

8. The medical device of claim 7, wherein the opening is disposed proximally of a distalmost end the cap.

9. The medical device of claim 7, wherein the gap terminates distally at a proximalmost surface of the cap.

10. The medical device of claim 1, wherein the sheath includes a tapered portion proximal of the opening.

11. The medical device of claim 1, wherein the fluid is a first fluid, and wherein the sheath includes a second fluid within a lumen of the sheath.

12. The medical device of claim 11, wherein compression of the sheath causes the second fluid to be ejected from the opening.

13. The medical device of claim 11, wherein compression of the sheath causes the medical device to transition from a first configuration to a second configuration, wherein the second configuration includes a smaller volume of the second fluid than the first configuration.

14. The medical device of claim 13 wherein, in the second configuration, at least a portion of the gap has a larger radial width than in the first configuration.

15. The medical device of claim 1, wherein the deformable member is a balloon.

16. A medical device comprising:
an outer tube;
a deformable member disposed radially within the outer tube;
a gap disposed radially between the outer tube and the deformable member; and
a sheath including a first fluid, wherein the sheath is radially within the deformable member;
wherein the gap is configured to receive a second fluid from a fluid source, wherein the second fluid is configured to deform the deformable member so that the deformable member compresses the sheath to deliver the first fluid through an opening of the sheath.

17. The medical device of claim 16, further comprising:
a hub, the hub including a fluid port that is configured to couple to the fluid source, wherein the fluid port is in fluid communication with the gap.

18. The medical device of claim 16, further comprising:
a cap coupled to a distal end of the outer tube and a distal end of the deformable member.

19. A medical device comprising:
a proximal hub including a fluid port; and
a shaft extending distally from the hub, wherein the shaft includes:
a balloon; and
a sheath containing a first fluid, wherein the sheath is disposed within a lumen of the balloon;
wherein delivery of a second fluid into the fluid port is configured to contact a radially outer surface of the balloon to cause the balloon to deform radially inward to compress the sheath and deliver the first fluid from an opening of the sheath.

20. The medical device of claim 19, wherein the shaft further includes an outer tube, wherein the balloon is disposed within the outer tube, wherein a gap is formed between the radially outer surface of the balloon and a radially inner surface of the outer tube, and wherein the fluid port is in fluid communication with the gap.
